(19) United States
(12) Patent Application Publication
(10) Pub. No.: US 2017/0231922 A1
(43) Pub. Date: Aug. 17, 2017

(54) 3, 5-DIHYDROXY-4-ISOPROPYLSTILBENE (DHIS) AS MITICIDAL AGENT AND ITS THERAPEUTIC USES

(71) Applicant: Horizon GenoMed Therapeutics, Inc, Vancouver (CA)

(72) Inventors: Youwen Zhou, Vancouver (CA); Cheng Xiao, Beijing (CN); Yang Wang, Beijing (CN); Yuanshen Huang, Vancouver (CA); Mingwan Su, Vancouver (CA)

(21) Appl. No.: 15/045,063

(22) Filed: Feb. 16, 2016

(51) Int. Cl. A61K 31/05; A61K 9/00; A01N 31/16

(52) U.S. Cl. CPC A61K 31/05; A01N 31/16; A61K 9/0014

(57) ABSTRACT

Disclosed herein are methods for treating skin conditions associated with skin parasites, including mites, by administering a therapeutically effective amount of 3,5-dihydroxy-4-isopropyl-trans-stilbene (DHIS).

### BACKGROUND

Technical Field

This disclosure relates to therapeutic use of 3,5-dihydroxy-4-isopropyl-trans-stilbene.

Description of the Related Art

3,5-Dihydroxy-4-isopropyl-trans-stilbene (DHIS) is a naturally occurring compound discovered many decades ago. It is a metabolic product of symbiotic microorganisms present in the soil. Its structure is shown below:

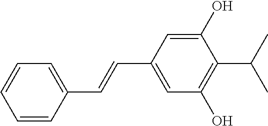 [SMILES: CC(C)c1c(O)cc(/C=C/c2ccccc2)cc1O]

DHIS is known to have antibacterial, anti-inflammatory, immune modulating, and anti-tumor activities. Although its exact and full biological activities are unknown, DHIS has been used as a topical agent in clinical trials for treating inflammatory skin diseases such as psoriasis and atopic dermatitis, with promising efficacy and minimal side effect. See e.g., Bissonnette et al. Arch Dermatol, 146(4): 446-449 (2010); Bissonnette et al. Br J Dermatol, 166(4): 853-860 (2012).

Due to its non-steroidal nature and ready availability through chemical synthesis, the therapeutic uses of DHIS would be significant. There is thus a need for exploring biological activities and effective therapeutic uses of DHIS.

### BRIEF SUMMARY

Provided herein are various embodiments directed to therapeutic use of 3,5-Dihydroxy-4-isopropyl-trans-stilbene (DHIS) as a miticidal agent.

One embodiment provides a method for treating skin affected by an overpopulation of skin parasites in a host, the method comprising contacting the affected skin with a therapeutically effective amount of DHIS.

A further embodiment provides a method for treating rosacea or acne in a subject in need thereof, the method comprising contacting the subject with a therapeutically effective amount of DHIS.

Yet another embodiment provides a method for eliminating mites or reducing mite population in a host, the method comprising contacting mites with 3,5-dihydroxy-4-isopropyl-trans-stilbene (DHIS).

### BRIEF DESCRIPTION OF THE SEVERAL VIEWS OF THE DRAWINGS

FIG. 1 shows the treatment results of individuals (N=10) having papulopustular rosacea.

FIG. 2 shows the treatment results of individuals (N=10) having acne.

FIGS. 3A-3B are photos of a rosacea patient prior to treatment and after 4 weeks of treatment using DHIS, respectively.

FIGS. 4A-4B are photos of an acne patient (cheek areas) prior to treatment and after 4 weeks of treatment using DHIS, respectively.

FIGS. 5A-5B are photos of an acne patient (forehead area) prior to treatment and after 4 weeks of treatment using DHIS, respectively.

### DETAILED DESCRIPTION

Described herein are therapeutic use of DHIS as an anti-parasitic agent and methods for treating skin conditions (e.g., rosacea and acne vulgaris) associated with skin parasites. In particular, it is disclosed herein that DHIS is a powerful miticidal agent for reducing or eradicating demodex mites.

### I. Skin Parasites and Associated Skin Conditions

Demodex mites are commensal ectoparasites of the human skin. There are two main types, including D. folliculorum and D. bravis. On the skin they typically are present in low numbers (0.7 mites/cm²) and reside in the hair follicles. Jarmuda et al, J Med Microbiol, 61(Pt 11): 1504-1510 (2012). They usually prefer areas of the skin with rich sebaceous glands, such as the face, forehead, nose, and the external ears. In these locations, the mites can mate and reproduce. The life span of demodex mites ranges 14-18 days. The propagation rate depends on the successful mating of the adult mites, which normally occur at the follicular openings of the sebaceous glands located at the surface of the skin. After mating, the female mites retreat back into the sebaceous glands located just below the surface of the epidermis, where they lay their eggs and start the life cycle of the next generation.

Studies have shown that low numbers of these organisms are present in the human skin, especially the face. The density of demodex mites is very low in young children. They start to increase in number around puberty, and continue to increase into adulthood.

In several skin conditions, such as rosacea, acne vulgaris, and blepharitis, perioral dermatitis, and alopecia, the mite density can be drastically increased compared to those without the skin conditions. In a survey of 860 individuals, individuals with normal facial skin only had a 5% chance to have mite density greater than 5/cm², as compared with 38.5% chance for rosacea and 9.3% chance for acne rosacea. Zhao et al. J Zhejiang Univ Sci B, 12(12): 1008-1015 (2011). It has also been disclosed that normal facial skin had a density of 0.7 mites/cm², as compared with 10.8 mites/cm² for rosacea. Forton et al. Br J Dermatol, 128(6): 650-659 (1993). In other studies, in addition to rosacea, several other skin conditions have been shown to be causally associated with increased infestation of the demodex mites, including blepharitis, and hair loss. Garcia-Vargas et al. J Am Acad Dermatol 57(2 Suppl): S19-21 (2007).

It is believed that while low density of mites in the skin is harmless to the skin, increased density of mites can be detrimental to the health of the skin. It has been concluded that when demodex mites breach the epithelial barrier, their antigens influence the immune system of the host and induce a type IV hypersensitivity reaction. Jarmuda (Supra).

Rosacea and acne vulgaris are described in further detail below.

1. Rosacea

Rosacea is a common facial disease that affects about 6% of the general population. The affected individuals develop redness of the skin, dilated superficial blood vessels, papules, pustules on the cheeks, nose, and forehead. In some people, the eyes may also be affected, especially blepharitis. In other patients, especially males, hypertrophy of the nose can develop, resulting in rhinophyma.

The exact etiology and pathogenesis of rosacea has not been clearly understood. Prevailing theories speculate on the role of vascular hypersensitivity versus increased immune activation.

High density of demodex mites is pathogenically linked to rosacea. Studies have shown vastly increased demodex mite in the rosacea skin, at a density of 10.7 mites/cm², at least 13 times higher than that of normal facial skin at a density of 0.7/cm². Jarmuda et al (Supra). Rosacea does not have an ideal therapy that is both effective and free of side effects. The current options include oral anti-inflammatory medications (e.g., doxycyclines) and systemic isotretinoins. However, the effects of these agents are temporary and are often associated with adverse events such as teratogenesities, allergic responses, GI intolerance, as well as photosensitivities, among others. Accordingly, these therapies are limited in their long-term clinical usefulness and safety. Topical treatments also exist, including metronidazoles (a miticide), ivermectin (a miticide), and precipitated sulphur (miticides and anti-inflammatory). However, these drugs are highly irritating to the skin, and their effects are mild to moderate.

2. Acne Vulgaris

Acne vulgaris is another follicular based skin disease centered on the face, chest, and upper back. The manifestations are similar to rosacea in terms of development of papules, pustules. However, acne patients tend to be much younger, and they do not typically develop facial flushing or dilated blood vessels. The pathogenesis is also unclear, although increased sebum production and blocked hair follicular openings are reported. Most recently multiple studies have shown that there is significantly increased density of demodex mites. In particular, a higher percentage of acne patients have significant demodex density compared to those without acne. Zhao et al. (supra).

Treatment of acne involves either systemic therapies or topical therapies. Systemic therapies for acne are similar to rosacea. However, the topical therapies are different, including retinoic acids, benzoyl peroxides, antibiotics. These treatments are either modestly effective, or are associated with significant side effects. Thus new therapies that are both effective and safe are needed.

### II. Miticidal Activity of DHIS

Various embodiments are directed to the miticidal activity of DHIS, which is proven effective in treating skin conditions such as rosacea and acne vulgaris.

DHIS has potent miticidal activities. As described herein, an in vitro miticidal assay was performed to determine the miticidal activities of DHIS by using human demodex mites prepared with pressure scraping method, following by microscopic examination of the survival time of demodex under microscopy, using the method described in Chinese Journal of Parasitology and Parasitic Diseases 2011; 29:258-263. As further demonstrated in Example 1, DHIS showed dose-dependent miticidal activity.

As used herein, mites include all demodex mites, such as human demodex folliculorum, D. bravis, and zoophilic demodex, such as D. canis, D. bovis, D. equi, D. ovis, D. cati, D. phyloides, and D. caprae. The demodex species include, without limitation, D. folliculorum and D. brevis.

Thus, one embodiment provides a method for eliminating mites or reducing mite population comprising contacting mites with DHIS.

In various specific embodiments, the mites are present on the skin of a host (e.g., human), including face, chest or upper back. In more specific embodiments, the mites are present on the skin at a density of more than 0.7 mite/cm², or more than 1.0 mite/cm², or more than 2.0 mites/cm², or more than 5.0 mites/cm², or more than 8.0 mites/cm², or more than 10 mites/cm², or more than 15 mites/cm².

### III. Treatment of Skin Diseases

Demodex mites are involved in the pathogenesis of several skin conditions, including rosacea and acne vulgaris. Consistent with the miticidal activities of DHIS observed in vitro, topical application of DHIS is further demonstrated to be effective in treating skin conditions (e.g., rosacea and acne vulgaris) associated with increased demodex mite. See Examples 2-5.

Therefore, various embodiments provide methods for treating skin affected by an overpopulation of skin parasites in a host, wherein the method comprises contacting the affected skin with a therapeutically effective amount of DHIS.

As used herein, a host refers to a human or an animal.

In preferred embodiments, the skin parasites are mites. In other embodiments, the skin parasites are pediculosis (lice).

As used herein, population or density of skin parasites (e.g, mites) refers to a number of parasites per unit area of skin surface. Typically, at a density of 0.7 mite/cm² or less, mites are harmless or nonpathogenic to the host. An increase in density from such a normal level (i.e., more than 0.7 mite/cm²) can lead to overpopulation or over-infestation of mites.

In addition to rosacea, acne vulgaris, skins affected by overpopulation or over-infestation of mites are associated with conditions such as ocular demodex infestation, blepharitis, Meibomian glandular abnormalities, chronic conjunctivitis, allergic conjunctivitis, scabies, animal scabies, animal demecidosis.

In various embodiments, the DHIS can be administered by various means, including IV, oral, perrectum, sublingual, topical, and intraocular.

If used as an oral agent, the DHIS can be formulated into tablets or capsules, which can be manufactured using non-medicinal materials such as starch, galactose, lubricants, humectants and so on. Oral application may also take the form of liquid or suspension, which may further include additives known in the art.

In a preferred embodiment, DHIS is directly applied to the skin, e.g., directly to the affected skin. DHIS may be formulated into creams, lotions, ointment, suspension, adhesives, foams, sprays, cleansers, gels.

In more specific embodiments, percutaneous or topical formulations may include one or more dermatologically acceptable excipients or additives to facilitate the per-cutaneous delivery (i.e., absorption through the skin). DHIS is typically present in the topical formulation at a concentration from approximately 0.2% to 20%, and any percentage or range in between. The compounded product or topical formulation may be applied in the amount of 0.5 mg to 1 gram. It is understood that the exact amounts or concentrations of DHIS may vary in individual circumstances depending on the severity of the skin condition.

When used for the treatment of rosacea, acne vulgaris as well as blepharitis, DHIS can be prepared as a 0.1% to 20% w/w compounded substance, applied to the affected area, once to twice per day, for as long as it takes to achieve clinical improvement or resolution of the symptoms.

DHIS may also be formulated as an instant release, delayed release or sustained release formula.

DHIS can be used in the same manner for the treatment of animal demodex infestations.

### EXAMPLES

### Example 1

### In Vitro Miticidal Activity of DHIS

In vitro miticidal assay was performed using human demodex mites prepared with pressure scraping method, following by microscopic examination of the survival time of demodex under microscopy, using the method described in Chinese Journal of Parasitology and Parasitic Diseases (Supra).

After mite isolation, 30 mites were placed on glass slides containing 200 ul of normal saline or saline containing various concentrations of DHIS (10 μM, 100 μM, and 1 mM). The slides were incubated at room temperature (20° C.) and under 70% humidity. The slides were examined under microscopy every 60 minutes for 8 hours after treatment. At each time point, the percentage of dead mites (no movements for 1 minute) was recorded for each treatment concentration. Each concentration was tested in triplicates, with the average for the three experiments recorded and shown in Table 1.

TABLE 1
Miticidal activities of DHIS in vitro

| | Number (%) mites dead at various time points | | | | | | | | | |
|---|---|---|---|---|---|---|---|---|---|---|
| | Demodex sp. | | | | | | | | | |
| DHIS | D. folliculorum | | | | | D. bravis | | | | |
| concentration | 0 hr | 1 hr | 2 hr | 3 hr | 4 hr | 0 hr | 1 hr | 2 hr | 3 hr | 4 hr |
| 0 | 0 | 0 | 0.3 | 1 | 3.3 | 0 | 0 | 0 | 1 | 2 |
| | | (0) | (1) | (3.3) | (11) | | (0) | (0) | (3.3) | (6.7) |
| 1 μM | 0 | 3 | 5 | 8.6 | 13 | 0 | 2 | 4 | 7 | 10 |
| | | (10) | (17) | (28) | (43) | | (6.7) | (13) | (23) | (33) |
| 10 μM | 0 | 7 | 12.3 | 14 | 22 | 0 | 6 | 11 | 14 | 19 |
| | | (23) | (41) | (47) | (73) | | (20) | (37) | (47) | (63) |
| 100 μM | 0 | 10 | 21.7 | 26 | 27.7 | 0 | 12 | 19 | 24 | 26 |
| | | (33) | (72) | (87) | (92) | | (40) | (63) | (80) | (87) |
| 1.0 mM | 0 | 13 | 24 | 28 | 29 | 0 | 14 | 18 | 26 | 26 |
| | | (43) | (80) | (93) | (97) | | (47) | (60) | (87) | (87) |

### Example 2

### DHIS Decreased Demodex Mites and Skin Manifestations of Patients with Papulopustular Rosacea

Given the demonstrated miticidal effect of DHIS in vitro, an observation was performed to examine (1) if topical application of DHIS on the skin of rosacea (which is well documented to have increased demodex mites) could result in decrease or eradication of demodex mites, and (2) if this is accompanied with improvement or remission of the clinical symptoms of rosacea.

A total of 20 volunteers (10 with papulopustular rosacea, 10 with papulopustular acne vulgaris) were recruited for this observation.

For each volunteer, the duration of the condition, the baseline sign and symptoms of their facial conditions and the severity of their condition were recorded. For the signs of the condition, papules and pustules were counted as total number for each individual. Erythema was rated as severe (3, deep red to purple), moderate (2, red), mild (1, pink), or none (0, normal color). For burning and pruritus, visual analogue scale (10 cm line) was used, with the score recorded from 0 (no symptoms) to 10 cm (most severe). In addition, the baseline demodex mite density of the perinasal skin was measured using the modified pressure-scraping technique. For the purpose of this study, both D. folliculorum and D. bravis were counted together. Then each volunteer used DHIS topical cream at 0.75% concentration, twice daily to the affected areas on the face. They were seen again at 4 weeks, when the clinical signs and symptoms were recorded. Further the demodex mite density was assessed again using the modified pressure/scraping technique.

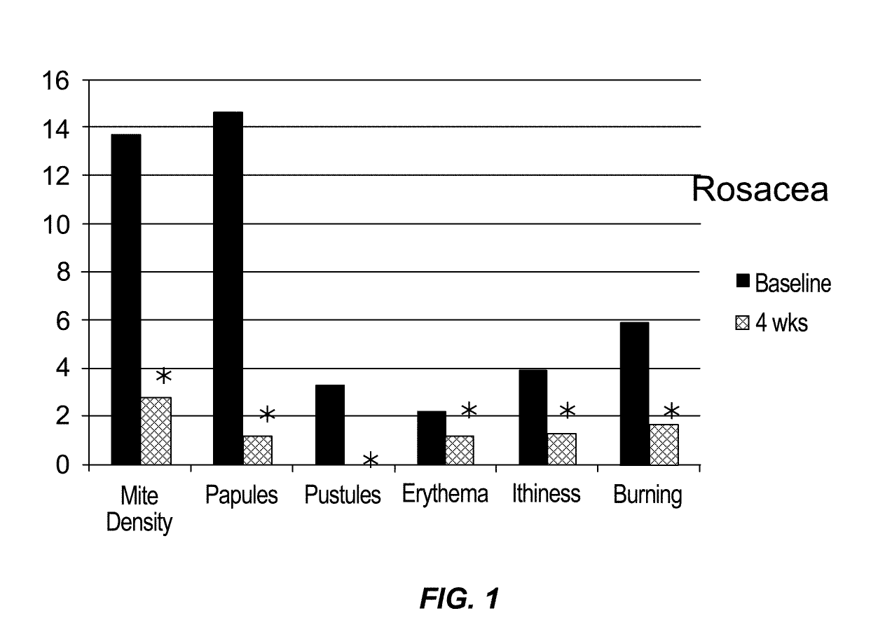

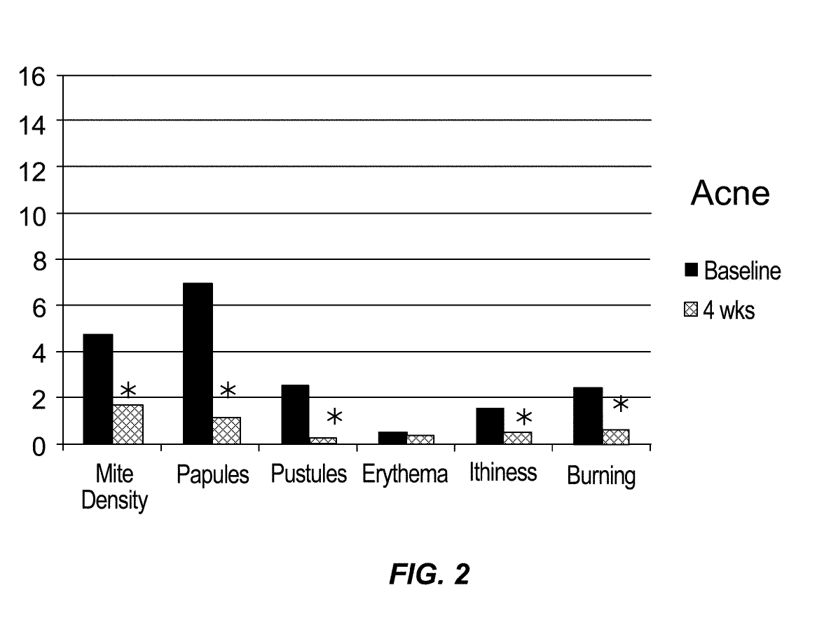

Individuals with papulopustular rosacea (FIG. 1, N=10) and acne (FIG. 2, N=10) received DHIS 0.75% cream twice daily for 4 weeks. The demodex mite density (mite/cm²) and clinical signs/symptoms are assessed at the baseline and again at 4 weeks. Papules and pustules are counted or the entire facial area for each individual. Erythema was graded as mild (1, pink), moderate (2, red), or severe (3, purple-red), whereas itchiness and burning sensation were graded according to a 10 cm visual analogue scale. * denotes p<0.05 (t-test)

As shown in FIG. 1, there was a significant reduction of demodex mite density at 4 weeks compared with at the baseline. Likewise, FIG. 2 shows a significant reduction of demodex mite density at 4 weeks compared with at the baseline for acne patients. The reduction of mite density was associated with dramatic reduction of lesional counts for papules and pustules, both for rosacea and for acne patients. The reduced erythema was also statistically significant. Other symptoms such as itchiness and burning sensation were reduced.

### Example 3

### Case Study—Rosacea Patient

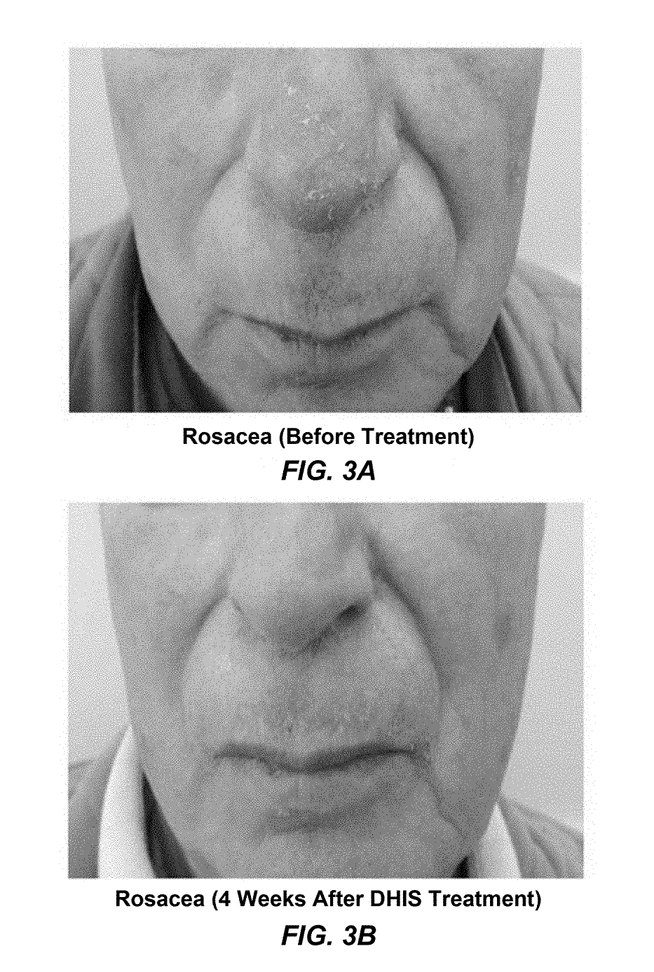

Case 1 involved a 76-year old man with rosacea for 11 years. He had facial erythema and papules and pustules, with mild telangiectasia. There was burning sensation. This was associated with mild rhinophyma. There was foreign body sensation in the eyes. He had received tetracycline and sulfonamide based creams with no obvious improvement. At base line, there was more than usual number of demodex mites. He received DHIS 0.75% cream treatment BID for 4 weeks. At the end of the therapy, his demodex mite density markedly decreased. More importantly, rosacea signs and symptoms dramatically improved. See photos of the rosacea patient prior to treatment (FIG. 3A) and after 4 weeks of treatment (FIG. 3B).

### Example 4

### Case Study—Acne Patient

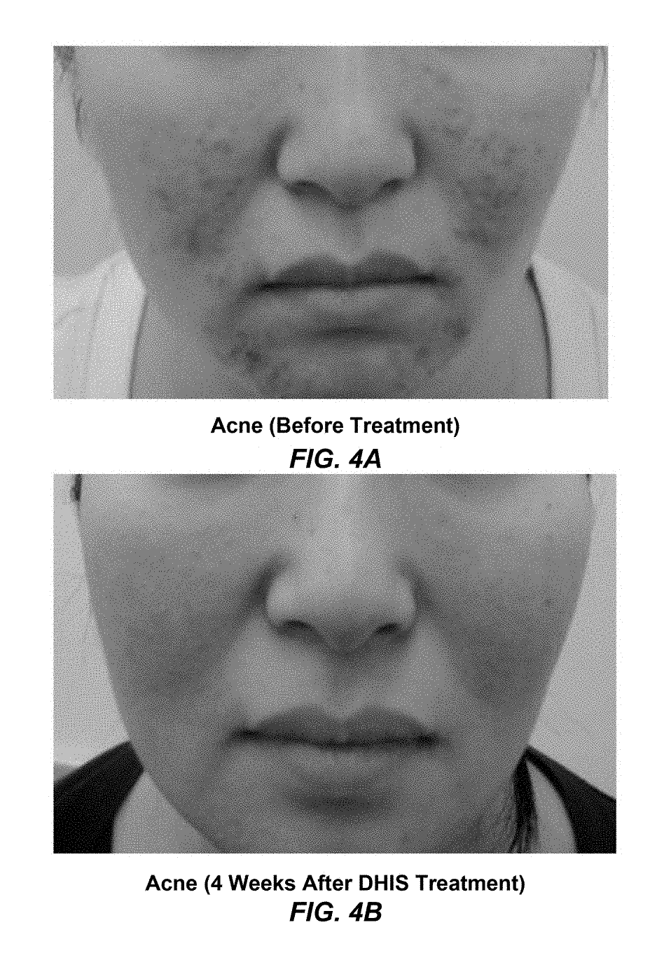

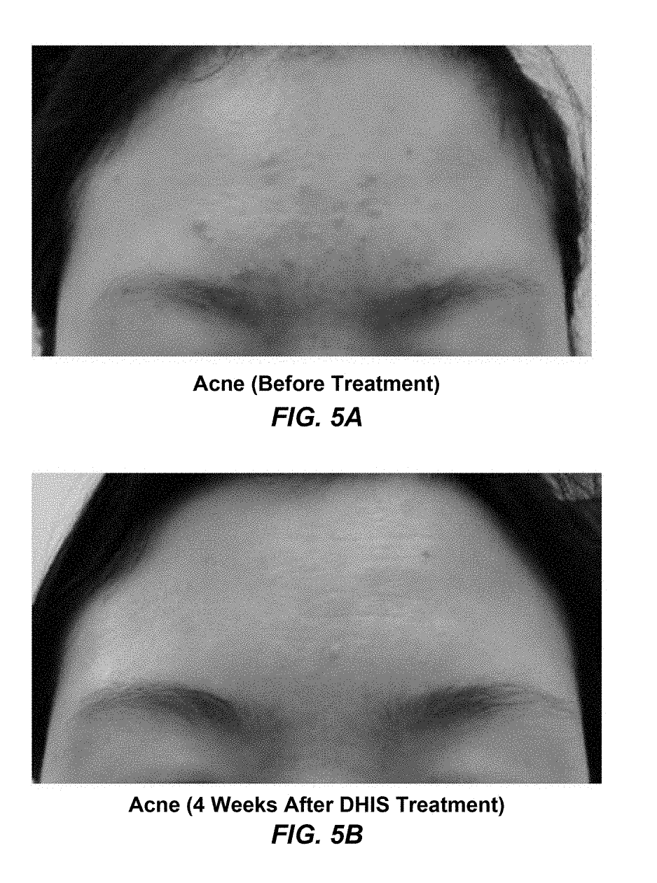

Case 2 involved a 21 year old woman with more than 3 years of acnes symptoms. She had open comedons as well as closed comedons. There were papules and pustules but only mild erythema. She had received topical retinoic acid therapy with no improvement. In addition, she received benzoyl peroxide/antibiotic combination topical therapy, oral birth control pills, oral tetracycline, as well as oral Traditional Chinese Medicine concoction, all without satisfactory effects. She had increased demodex mite count at base line. After receiving 4 weeks of DHIS 0.75% cream BID, her facial demodex mite density significantly decreased. Her facial acne papules and pustules resolved at the end of 4 weeks. See photos of the acne patient (cheeks and forehead) prior to treatment (FIGS. 4A and 5A) and after 4 weeks of treatment (FIGS. 4B and 5B).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating skin affected by an overpopulation of skin parasites in a host, the method comprising contacting the affected skin with a therapeutically effective amount of 3,5-dihydroxy-4-isopropyl-trans-stilbene (DHIS).

2. The method of claim 1 wherein the skin parasites are demodex mites.

3. The method of claim 2 wherein the demodex mites are present at density of more than 0.7 mite/cm².

4. The method of claim 2 wherein the demodex mites are present at density of more than 5 mites/cm².

5. The method of claim 2 wherein the demodex mites are present at density of more than 10 mites/cm².

6. The methods of claim 1 wherein the host is human or an animal.

7. The method of claim 1 wherein the host has rosacea on the affected skin.

8. The method of claim 1 wherein the host has acne on the affected skin.

9. A method for treating rosacea or acne in a subject in need thereof, the method comprising contacting the subject with a therapeutically effective amount of 3,5-dihydroxy-4-isopropyl-trans-stilbene (DHIS).

10. A method for eliminating mites or reducing mite population in a host, the method comprising contacting mites with 3,5-dihydroxy-4-isopropyl-trans-stilbene (DHIS).

11. The method of claim 10, wherein the mites are present on the host's skin.

12. The method of claim 10 wherein the mites are present on the host's skin at density of more than 0.7 mite/cm².

13. The method of claim 10 wherein the mites are present on the host's skin at density of more than 5.0 mites/cm².

14. The method of claim 10 wherein the mites are present on the host's skin at density of more than 10 mites/cm².
